(19) United States
(12) Patent Application Publication
(10) Pub. No.: US 2026/0116936 A1
(43) Pub. Date: Apr. 30, 2026

(54) MYOSTATIN PROTEIN MUTANT AND PREPARATION METHOD THEREOF

(71) Applicant: Jiangsu Academy of Agricultural Sciences, Nanjing City (CN)

(72) Inventors: Shaoxian CAO, Nanjing City (CN); Huili WANG, Nanjing City (CN); Yinxia LI, Nanjing City (CN); Rihong GUO, Nanjing City (CN); Chunhua MENG, Nanjing City (CN); Zhaokang CUI, Nanjing City (CN); Qiang DING, Nanjing City (CN); Yong QIAN, Nanjing City (CN); Kunlin CHEN, Nanjing City (CN); Jianli ZHANG, Nanjing City (CN); Jun ZHANG, Nanjing City (CN)

(21) Appl. No.: 19/345,494

(22) Filed: Sep. 30, 2025

Related U.S. Application Data
(63) Continuation of application No. PCT/CN2025/078189, filed on Feb. 20, 2025.

(30) Foreign Application Priority Data
Dec. 21, 2023 (CN) 202311771982.X

(51) Int. Cl. C07K 14/47; C12N 15/11

(52) U.S. Cl. CPC C07K 14/4716; C12N 15/111

(57) ABSTRACT

A myostatin (MSTN) protein mutant and a preparation method thereof are provided. The amino acid sequence of the mutant is any one of the sequences shown in SEQ ID NO. 15, SEQ ID NO. 17 or SEQ ID NO. 20. Under the guidance of specially designed single guide ribonucleic acid (sgRNA), a mutant in which the 281st and/or 282nd cysteine of the sheep myostatin full length protein containing the signal peptide is mutated into arginine is obtained through adenine base editor (ABE) editing, and the nucleotide sequence of the sgRNA is shown in SEQ ID NO. 5.

### CROSS-REFERENCE TO RELATED APPLICATIONS

This application is a continuation of International Patent Application No. PCT/CN2025/078189, filed on Feb. 20, 2025 and claims priority to Chinese Patent Application No. 202311771982.X, filed on Dec. 21, 2023. The contents of International Patent Application No. PCT/CN2025/078189 and Chinese Patent Application No. 202311771982.X are hereby incorporated by reference.

### INCORPORATION BY REFERENCE STATEMENT

This statement, made under Rules 77(b)(5)(ii) and any other applicable rule incorporates into the present specification of an XML file for a “Sequence Listing XML” (see Rule 831(a)), submitted via the USPTO patent electronic filing system or on one or more read-only optical discs (see Rule 1.52(e)(8)), identifying the names of each file, the date of creation of each file, and the size of each file in bytes as follows:
File name: SequenceListing.xml
Creation date: Sep. 29, 2025
Byte size: 32,682

### TECHNICAL FIELD

The disclosure relates to the fields of genetic engineering and animal husbandry genetics and breeding, and in particular to a myostatin (MSTN) protein mutant and a preparation method thereof.

### BACKGROUND

Myostatin (MSTN), a member of transforming growth factor-β (TGF-β) superfamily, is mainly expressed in skeletal muscle and is a negative regulator of muscle growth and development. Over-expression of myostatin gene in mice inhibits the proliferation and differentiation of skeletal muscle, a single muscle cell grows to about twice the normal size after knockout, and the number of muscle fibers increases significantly, showing a double muscling phenomenon, and the weight increases significantly. A myostatin gene of sheep consists of 3 exons and 2 introns, the full length of a coding sequence (CDS) region is 1128 nucleotides (nt), and the myostatin gene of sheep is translated into a protein containing 375 amino acid residues. The myostatin protein has a typical TGF-β superfamily structure, including a signal peptide with 24 amino acid residues, an N-terminal propeptide and a mature peptide with 9 Cysteine residues at the C-terminal. The maturation of myostatin protein goes through three digestion processes: signal peptide cleavage, serine kinase Furin digestion and BMP-1/TLD metalloproteinases digestion. The myostatin protein signal peptide is hydrolyzed to form pro-myostatin, and two pro-myostatin forms homodimer through intermolecular disulfide bonds. Serine kinase Furin recognizes and cleaves the arginine-serine-arginine-arginine site (amino acid residues 263-266), forming a 27.7 kilodalton (kDa) propeptide and a 12.5 kDa mature peptide, which are released to the extracellular space and play a role through internal secretion or paracrine secretion. Most of the myostatin released to the extracellular space exists in the form of Latent-myostatin, namely the propeptide of myostatin and mature peptide are bound together through non-covalent bonds, after the Latent-myostatin is transported to the target site, BMP-1/TLD metalloproteinases cleave myostatin precursor (amino acid residue 98th) and release active myostatin mature peptide dimer, myostatin mature peptide contains 9 Cysteine residues, and the Cysteine residues form active myostatin with advanced structure through 4 pairs of intramolecular disulfide bonds and 1 pair of intermolecular disulfide bonds.

Myostatin is highly conserved in mammals, mutation or deletion of nucleotide may lead to inactivation or decreased activity of myostatin protein, which may lead to obstruction of downstream signal pathway of myostatin, activation, proliferation and differentiation of skeletal muscle satellite cells, and increase of myofibrillar protein synthesis, and finally manifest as hypertrophy or proliferation of muscle fibers. The deletion mutation of the coding region of myostatin gene in Belgian Blue led to the early termination of protein translation, resulting in the formation of truncated myostatin protein and the appearance of double hindquarters muscling phenotype. The myostatin messenger ribonucleic acid (mRNA) level of Texel sheep myostatin 3′-UTR with SNP c.*1232G>A mutation did not change, but the myostatin protein level in blood decreased by 66%, showing an obvious double muscling phenotype. The Whippet dog breed carrying the myostatin c.939(del2) mutation exhibits the typical double muscling phenotype.

In view of this, the researchers try to create double muscling phenotype livestock by myostatin gene knockout, so as to provide germplasm resources for livestock breeding. Using CRISPR/Cas9 gene editing technology may occur mutations in the gene coding region, result amino acid addition, deletion or substitution, so as to achieve gene function knockout or knockdown. For example, Guo R et al. edited the myostatin gene of Haimen goat by using CRISPR/Cas9, and obtained goat individuals with double muscling phenotype; Wang X et al. edited myostatin gene of Tan sheep by using CRISPR/Cas9, and also obtained sheep individuals with double muscling phenotype; a patent of Lian Zhengxing et al. (CN 111793123 A) obtained a mutant of sheep myostatin by using CRISPR/Cas9, namely the 339th cysteine deletion of myostatin full length protein containing signal peptide, and the animals carrying this mutation have obvious double-muscled hindquarters characteristics. In the above examples, the sheep with double muscling phenotype are all created by insertion-deletion (Indel), which is generated through editing the myostatin by using CRISPR/Cas9. Due to the high randomness and complexity of genotypes of CRISPR/Cas9 editing in generating indel types, only a subset or even a minor fraction of these indel types achieve the intended goal of gene knockout or knockdown. Therefore, although the editing efficiency of CRISPR/Cas9 has made great progress compared with the previous editing methods, the efficiency of gene function knockout or knockdown is still uncertain. Considering the high cost and long cycle of large animals, it is still necessary to use more accurate gene editing methods to efficiently create mutants with double muscling phenotype.

### SUMMARY

The objective of the disclosure is to provide a myostatin (MSTN) protein mutant and a preparation method thereof, so as to solve the problems existing in the prior art. In the disclosure, under the guidance of specially designed single guide ribonucleic acid (sgRNA), adenine base editor (ABE) editing induces T-to-C mutations at positions 841st, 844th, and 846th in the coding region of the full-length myostatin gene (Gene ID: 443449) containing a signal peptide. This results in the formation of a mutant full-length myostatin protein (NP_001009428.1) with cysteine-to-arginine substitutions at positions 281 and/or 282, which disrupts the intramolecular disulfide bonds of myostatin. Consequently, the formation of active myostatin with specific higher-order structure is impeded, thereby achieving myostatin inactivation. Animals carrying this mutant exhibit distinct double-muscled phenotypes, which provides technical support for efficient production of double-muscled sheep and breed development.

In order to achieve the above objective, the disclosure provides the following scheme:

The disclosure provides a myostatin protein mutant, the amino acid sequence of which is any one of the sequences shown in SEQ ID NO. 15, SEQ ID NO. 17 or SEQ ID NO. 20.

The disclosure also provides a gene encoding the myostatin protein mutant.

In an embodiment, when the amino acid sequence of the myostatin protein mutant is SEQ ID NO. 15, the nucleotide sequence of the gene is shown in SEQ ID NO. 13; when the amino acid sequence of the myostatin protein mutant is SEQ ID NO. 17, the nucleotide sequence of the gene is shown in SEQ ID NO. 16 or SEQ ID NO. 18; and when the amino acid sequence of the myostatin protein mutant is SEQ ID NO. 20, the nucleotide sequence of the gene is shown in SEQ ID NO. 19 or SEQ ID NO. 21.

The disclosure also provides a method for preparing the biological material including the above mutants in its genome, including a step of obtaining the biological material through ABE editing guided by an sgRNA; and
where the nucleotide sequence of the sgRNA is shown in SEQ ID NO. 5.

In an embodiment, the biological material includes cells, embryos and animals.

The disclosure also provides an sgRNA for editing myostatin gene, and the nucleotide sequence of the sgRNA is shown in SEQ ID NO. 5.

The disclosure also provides an expression vector or expression cassette for expressing the sgRNA.

The disclosure also provides a method for improving animal muscle content or promoting animal muscle development, which includes utilizing the sgRNA or the expression vector or the expression cassette to mutate the myostatin coding gene in the animal genome into the above gene through ABE editing, where the animal is a sheep.

The disclosure also provides a method for improving the meat yield of animals, which includes utilizing the sgRNA or the expression vector or the expression cassette to mutate the myostatin coding gene in the animal genome into the above gene through ABE editing, where the animals are sheep.

The disclosure also provides a genetic breeding method for animals of double hindquarters muscling, which includes mutating the myostatin coding gene in the animal genome into the above gene, where the animals are sheep.

It should be understood by the skilled in the art that, based on the conservation and homology of myostatin proteins across different animal species, myostatin protein mutants and their encoding genes of other animals obtained through homologous alignment, wherein the mutant proteins contain either or both of a cysteine-to-arginine mutation at position 281st corresponding to SEQ ID NO:15, and/or a cysteine-to-arginine mutation at position 282nd corresponding to SEQ ID NO:17, are also within the protection scope of the disclosure, provided that they exhibit biological functionality equivalent to the above mutants described herein. This protection extends to all orthologous variants exhibiting comparable myostatin-inhibitory activity resulting from these specified amino acid substitutions at conserved positions across species.

The disclosure discloses the following technical effects.

According to the disclosure, through the ABE editing, a mutant in which the 281st and/or 282nd cysteine of the sheep myostatin full length protein containing the signal peptide is mutated into arginine is obtained, the mutant may promote the growth and development of sheep muscles, and the cross-sectional area of sheep muscle fibers carrying the myostatin mutant of the disclosure is increased, showing an obvious double hindquarters muscling phenotype, thereby increasing the meat yield.

The disclosure provides an sgRNA which efficiently targets the myostatin protein coding gene sequence, under the guidance of the sgRNA, through ABE editing technology, people may successfully obtain the mutant that the cysteine at the 281st and/or the cysteine at the 282nd position of the sheep myostatin full length protein containing signal peptide is mutated into arginine, and the editing efficiency is over 70%; and under the guidance of the sgRNA, utilizing the ABE editing technology, animal models with double hindquarters muscling phenotype may be efficiently prepared or genetic breeding of double muscling hindquarters traits may be realized.

### BRIEF DESCRIPTION OF THE DRAWINGS

In order to more clearly explain the technical scheme in the embodiment of the present disclosure or in the prior art, the drawings needed in the embodiment will be briefly introduced below, obviously, the drawings in the following description are only some embodiments of the disclosure, and other drawings may be obtained according to these drawings without creative work for ordinary people in the field.

FIG. 1A and FIG. 1B are the genotype sequencing analysis of sheep embryos carrying the coding genes of the 281st and/or 282nd mutation of myostatin (MSTN) full length protein edited and prepared by adenine base editor (ABE) editing; FIG. 1A is of WT, MT1, MT2 and MT3, where WT is wild type, MT1 is a mutant in which the 841st T of the coding region of myostatin full length gene (Gene ID:443449) containing signal peptide is partially substituted to C (chimeric), and then forming a mutant in which the 281st cysteine of myostatin full length protein (NP_001009428.1) containing signal peptide is partially substituted to arginine, MT2 is a mutant in which the 844th T of the coding region of myostatin full length gene (Gene ID:443449) containing signal peptide is partially substituted to C (chimeric), and then forming a mutant in which the 282nd cysteine of myostatin full length protein (NP_001009428.1) containing signal peptide is partially substituted to arginine, and MT3 is a mutant in which both the 844th and 846th T in the coding region of myostatin full length gene (Gene ID: 443449) containing signal peptide are partially substituted to C (chimeric), and then forming a mutant in which the 282nd cysteine of myostatin full length protein (NP_001009428.1) containing signal peptide is partially substituted to arginine. FIG. 1B is of MT4, MT5, MT6 and MT7, where MT4 is a mutant in which both the 844th and 846th T in the coding region of myostatin full length gene (Gene ID: 443449) containing signal peptide are mutated into C, and then forming a mutant in which the 282nd cysteine of myostatin full length protein (NP_001009428.1) containing signal peptide is mutated into arginine; MT5 is a mutant in which both the 841st and 844th T in the coding region of myostatin full length gene (Gene ID: 443449) containing signal peptide are mutated into C, and then forming a mutant in which the 281st and 282nd cysteine of myostatin full length protein (NP_001009428.1) containing signal peptide are mutated into arginine; MT6 is a mutant in which the 841st, 844th and 846th T in the coding region of myostatin full length gene (Gene ID: 443449) containing signal peptide are mutated into C, and then forming a mutant in which the 281st and 282nd cysteine of myostatin full length protein (NP_001009428.1) containing signal peptide are mutated into arginine; and MT7 is a mutant in which the 844th T in the coding region of myostatin full length gene (Gene ID: 443449) containing signal peptide is mutated into C, and the 841st and 846th T are partially substituted to C, and then forming a mutant in which the signal peptide-containing full-length myostatin protein (NP_001009428.1) harbors the 282nd cysteine residue completely mutated to arginine and the 281st cysteine residue partially substituted by arginine.

FIG. 2 is the genotype sequencing analysis of Hu sheep carrying the coding gene of the 281st and/or 282nd mutation of myostatin full length protein prepared by ABE editing; of which WT is a wild-type Hu sheep, MT1 is a Hu sheep in which the 282nd cysteine of myostatin full length protein is mutated into arginine; MT2 is a Hu sheep in which the 281st cysteine of myostatin full length protein is mutated into arginine; and MT3 and MT4 are Hu sheep whose cysteine at positions 281 and 282 of myostatin full length protein mutated into arginine.

FIG. 3 is the growth phenotype analysis of Hu sheep carrying myostatin protein mutant; mutant lambs are Hu sheep with myostatin protein mutant, and wild Hu sheep are used as control group.

FIG. 4 is a phenotypic comparison between mutant Hu sheep individuals (MT1, MT2 and MT3) carrying myostatin protein mutants and wild-type Hu sheep individuals (WT); of which A is MT1, carrying the 282nd mutant of myostatin full length protein, and the wild-type Hu sheep take pictures from the front perspective; B is MT1 and the wild Hu sheep take pictures from the side view; C is the Hu sheep individual MT2, carrying the 281st mutant of myostatin full length protein, takes picture from the side view; and D is the Hu sheep individual MT3, carrying the 281st and 282nd mutant of myostatin full length protein, takes the picture from the side view.

FIG. 5A, FIG. 5B are comparison of muscle fibers between mutants Hu sheep MT1 carrying the 282nd mutant of myostatin full length protein, MT2 carrying the 281st mutant of myostatin full length protein, MT3 carrying the 281st and 282nd mutants of myostatin full length protein and myostatin gene wild-type Hu sheep (WT); of which FIG. 5A is the comparison of HE staining results of muscle tissue slices (crosscutting), and FIG. 5B is the comparison of cross-sectional area of muscle fibers.

### DETAILED DESCRIPTION OF THE EMBODIMENTS

A number of exemplary embodiments of the disclosure will be described in detail now, and the detailed description should not be considered as a limitation of the disclosure, but should be understood as a more detailed description of certain aspects, characteristics and embodiments of the disclosure.

It should be understood that the terminology described in the disclosure is only for describing specific embodiments and is not used to limit the disclosure. In addition, for the numerical range in the disclosure, it should be understood that each intermediate value between the upper limit and the lower limit of the range is also specifically disclosed. Intermediate values within any stated value or stated range, as well as each smaller range between any other stated value or intermediate values within the stated range are also included in the disclosure. The upper and lower limits of these smaller ranges may be independently included or excluded from the range.

Unless otherwise specified, all technical and scientific terms used herein have the same meaning as commonly understood by one of ordinary skill in the art to which the disclosure relates. Although the disclosure only describes the preferred methods and materials, any methods and materials similar or equivalent to those described herein may also be used in the practice or testing of the disclosure. All documents mentioned in this specification are incorporated by reference to disclose and describe methods and/or materials related to the documents. In case of conflict with any incorporated document, the contents of this specification shall prevail.

It is obvious to those skilled in the art that many improvements and changes may be made to the specific embodiments of the disclosure without departing from the scope or spirit of the disclosure. Other embodiments will be apparent to the skilled person from the description of the disclosure. The description and example of that disclosure are exemplary only.

The terms “comprising”, “including”, “having” and “containing” used in this article are all open terms, which means including but not limited to.

### Embodiment 1 Adenine Base Editor (ABE) Editing Prepares Sheep Embryos Carrying the Coding Genes of the 281st and/or 282nd Mutant of Myostatin (MSTN) Full Length Protein

1. Construction of Single Guide Ribonucleic Acid (sgRNA) Expression Vector

According to the sequence of sheep myostatin gene as the target sequence, the oligonucleotide DNA sequence is designed and sent to a commercial primer synthesis company for synthesis (each strand is synthesized for about 1OD, and the purification method is PAGE). The specific sequence is as follows:

sg-F:
SEQ ID NO. 1
5′-CCGGACGACAGCATCGAGATTCTG-3′,;
sg-R:
SEQ ID NO. 2
5′-AAACCAGAATCTCGATGCTGTCGT-3′,.

The above two oligo DNA are dissolved in ultrapure water, mixed and annealed to form a double-stranded DNA fragment sg-FR with the sticky end of BsaI restriction site;

The recombinant vector pGL3-sg is constructed by ligating the sg-FR fragment into the linearized pGL3-U6-sgRNA-pgk-puromycin vector (Addgene), which has been digested with BsaI. This pGL3-sg vector serves as an sgRNA expression vector, in which the inserted sg-FR fragment is fused with the vector backbone to drive sgRNA expression.

2. In Vitro Transcription of sgRNA and ABE Messenger Ribonucleic Acid (mRNA)

Using recombinant vector pGL3-sg as a template, the primers sgRNA-TF with T7 promoter sequences at their 5′ ends (sequence: 5′-TAATACGACTCACTATAGGACGACAGCATCGAGATTCTG-3′, SEQ ID NO. 3) and sgRNA-TR (sequence: 5′-AAAAGCACCGACTCGGTGCCA-3′, SEQ ID No.4) are designed and synthesized for Polymerase Chain Reaction (PCR) amplification. The amplified product is gel-purified to obtain the T7 promoter-containing sgRNA in vitro transcription template. Subsequently, 200 nanogram (ng) of the purified template is used for sgRNA in vitro transcription following the manufacturer's protocol of the Ambion MEGAshortscript™ T7 Transcription Kit, yielding the final sgRNA product. The nucleotide sequence of sgRNA is as follows:

sgRNA (SEQ ID NO. 5):
5′-acgacagcaucgagauucugguuuuagagcuagaaauagcaaguu
aaaauaaggcuaguccguuaucaacuugaaaaaguggcaccgagucgg
ugc-3′,

The target sequence (5′-acgacagcatcgagattctg-3′, SEQ ID NO. 6) corresponds to the antisense strand of nucleotides 83-102 within exon 3 of the sheep myostatin gene (Gene ID: 443449).

Using ABE8e (provided by addgene) as a template, the primers ABE-F (sequence: 5′-CTAATACGACTCACTATAGGGAGAG-3′, SEQ ID NO. 7) with T7 promoter at the 5′ end and ABE-R (sequence: 5′-AAGGCACAGTCGAGGCTG-3′, SEQ ID NO. 8) are designed and synthesized. PCR amplification followed by gel purification yields the T7 promoter-containing ABE in vitro transcription template. Using 1000 ng of purified template, in vitro transcription (Vazyme, T7 High Yield RNA Transcription Kit, DD4201-01) and polyadenylation (Vazyme, E. coli Poly(A) Polymerase, DD4111-PC-01) are performed to generate the ABE mRNA (encoding sequence: SEQ ID NO. 9).

SEQ ID NO. 9:
augaaacggacagccgacggaagcgaguucgagucaccaaagaagaagcggaaagucucugagguggaguuuu
cccacgaguacuggaugagacaugcccugacccuggccaagagggcacgggaugagagggaggugccugugggagccgugcu
ggugcugaacaauagagugaucggcgagggcuggaacagagccaucggccugcacgacccaacagcccaugccgaaauuaugg
cccugagacagggcggccuggucaugcagaacuacagacugauugacgccacccuguacgugacauucgagccuugcgugau
gugcgccggcgccaugauccacucuaggaucggccgcgugguguuuggcgugaggaacucaaaaagaggcgccgcaggcucc
cugaugaacgugcugaacuaccccggcaugaaucaccgcgucgaaauuaccgagggaauccuggcagaugaaugugccgcccu
gcugugegauuucuaucggaugccuagacagguguucaaugcucagaagaaggcccagagcuccaucaacuccggaggaucu
agcggaggcuccucuggcucugagacaccuggcacaagcgagagcgcaacaccugaaagcagcgggggcagcagcggggggu
cagacaagaaguacagcaucggccuggccaucggcaccaacucugugggcugggccgugaucaccgacgaguacaaggugccc
agcaagaaauucaaggugcugggcaacaccgaccggcacagcaucaagaagaaccugaucggagcccugcuguucgacagcgg
cgaaacagccgaggccacccggcugaagagaaccgccagaagaagauacaccagacggaagaaccggaucugcuaucugcaaga
gaucuucagcaacgagauggccaagguggacgacagcuucuuccacagacuggaagaguccuuccugguggaagaggauaag
aagcacgagcggcaccccaucuucggcaacaucguggacgagguggccuaccacgagaaguaccccaccaucuaccaccugag
aaagaaacugguggacagcaccgacaaggccgaccugcggcugaucuaucuggcccuggcccacaugaucaaguuccggggcc
acuuccugaucgagggcgaccugaaccccgacaacagcgacguggacaagcuguucauccagcuggugcagaccuacaaccag
cuguucgaggaaaaccccaucaacgccagcggcguggacgccaaggccauccugucugccagacugagcaagagcagacggcu
ggaaaaucugaucgcccagcugcccggcgagaagaagaauggccuguucggaaaccugauugcccugagccugggccugacc
cccaacuucaagagcaacuucgaccuggccgaggaugccaaacugcagcugagcaaggacaccuacgacgacgaccuggacaac
cugcuggcccagaucggcgaccaguacgccgaccuguuucuggccgccaagaaccuguccgacgccauccugcugagcgacau
ccugagagugaacaccgagaucaccaaggccccccugagcgccucuaugaucaagagauacgacgagcaccaccaggaccugac
ccugcugaaagcucucgugcggcagcagcugccugagaaguacaaagagauuuucuucgaccagagcaagaacggcuacgccg
gcuacauugacggcggagccagccaggaagaguucuacaaguucaucaagcccauccuggaaaagauggacggcaccgaggaa
cugcucgugaagcugaacagagaggaccugcugeggaagcagcggaccuucgacaacggcagcaucccccaccagauccaccu
gggagagcugcacgccauucugcggcggcaggaagauuuuuacccauuccugaaggacaaccgggaaaagaucgagaagauc
cugaccuuccgcauccccuacuacgugggcccucuggccaggggaaacagcagauucgccuggaugaccagaaagagcgagga
aaccaucacccccuggaacuucgaggaagugguggacaagggcgcuuccgcccagagcuucaucgagcggaugaccaacuucg
auaagaaccugcccaacgagaaggugcugcccaagcacagccugcuguacgaguacuucaccguguauaacgagcugaccaaa
gugaaauacgugaccgagggaaugagaaagcccgccuuccugagcggcgagcagaaaaaggccaucguggaccugcuguuca
agaccaaccggaaagugaccgugaagcagcugaaagaggacuacuucaagaaaaucgagugcuucgacuccguggaaaucucc
ggcguggaagaucgguucaacgccucccugggcacauaccacgaucugcugaaaauuaucaaggacaaggacuuccuggacaa
ugaggaaaacgaggacauucuggaagauaucgugcugacccugacacuguuugaggacagagagaugaucgaggaacggcug
aaaaccuaugcccaccuguucgacgacaaagugaugaagcagcugaagcggcggagauacaccggcuggggcaggcugagccg
gaagcugaucaacggcauccgggacaagcaguccggcaagacaauccuggauuuccugaaguccgacggcuucgccaacagaa
acuucaugcagcugauccacgacgacagccugaccuuuaaagaggacauccagaaagcccagguguccggccagggcgauagc
cugcacgagcacauugccaaucuggccggcagccccgccauuaagaagggcauccugcagacagugaaggugguggacgagc
ucgugaaagugaugggccggcacaagcccgagaacaucgugaucgaaauggccagagagaaccagaccacccagaagggacag
aagaacagccgcgagagaaugaagcggaucgaagagggcaucaaagagcugggcagccagauccugaaagaacaccccgugga
aaacacccagcugcagaacgagaagcuguaccuguacuaccugcagaaugggcgggauauguacguggaccaggaacuggaca
ucaaccggcuguccgacuacgauguggaccauaucgugccucagagcuuucugaaggacgacuccaucgacaacaaggugcu
gaccagaagcgacaagaaccggggcaagagcgacaacgugcccuccgaagaggucgugaagaagaugaagaacuacuggcggc
agcugcugaacgccaagcugauuacccagagaaaguucgacaaucugaccaaggccgagagaggcggccugagcgaacuggau
aaggccggcuucaucaagagacagcugguggaaacccggcagaucacaaagcacguggcacagauccuggacucccggaugaa
cacuaaguacgacgagaaugacaagcugauccgggaagugaaagugaucacccugaaguccaagcugguguccgauuuccgg
aaggauuuccaguuuuacaaagugcgcgagaucaacaacuaccaccacgcccacgacgccuaccugaacgccgucgugggaac
cgcccugaucaaaaaguacccuaagcuggaaagcgaguucguguacggcgacuacaagguguacgacgugcggaagaugauc
gccaagagcgagcaggaaaucggcaaggcuaccgccaaguacuucuucuacagcaacaucaugaacuuuuucaagaccgagau
uacccuggccaacggcgagauccggaagcggccucugaucgagacaaacggcgaaaccggggagaucgugugggauaagggc
cgggauuuugccaccgugcggaaagugcugagcaugccccaagugaauaucgugaaaaagaccgaggugcagacaggcggcu
ucagcaaagagucuauccugcccaagaggaacagcgauaagcugaucgccagaaagaaggacugggacccuaagaaguacggc
ggcuucgacagccccaccguggccuauucugugcuggugguggccaaaguggaaaagggcaaguccaagaaacugaagagug
ugaaagagcugcuggggaucaccaucauggaaagaagcagcuucgagaagaaucccaucgacuuucuggaagccaagggcuac
aaagaagugaaaaaggaccugaucaucaagcugccuaaguacucccuguucgagcuggaaaacggccggaagagaaugcuggc
cucugccggcgaacugcagaagggaaacgaacuggcccugcccuccaaauaugugaacuuccuguaccuggccagccacuaug
agaagcugaagggcucccccgaggauaaugagcagaaacagcuguuuguggaacagcacaagcacuaccuggacgagaucauc
gagcagaucagcgaguucuccaagagagugauccuggccgacgcuaaucuggacaaagugcuguccgccuacaacaagcaccg
ggauaagcccaucagagagcaggccgagaauaucauccaccuguuuacccugaccaaucugggagccccugccgccuucaagu
acuuugacaccaccaucgaccggaagagguacaccagcaccaaagaggugcuggacgccacccugauccaccagagcaucaccg
gccuguacgagacacggaucgaccugucucagcugggaggugacucuggcggcucaaaaagaaccgccgacggcagcgaauu
cgagcccaagaagaagaggaaagucuaa.

### 3. Microinjection of Sheep Embryos

Sheep fertilized eggs are subjected to cytoplasmic microinjection using a mixed system of prepared sgRNA and ABE mRNA (sgRNA+ABE mRNA). The final concentrations in the mixed system are 50 ng/μL for sgRNA and 100 ng/μL for ABE mRNA. Fertilized eggs are injected with 5 μL of the mixed system per egg and cultured in vitro to the blastocyst stage.

### 4. Single-Blastocyst Whole-Genome Amplification (WGA)

The single sheep blastocyst obtained above is taken into a PCR tube, phosphate buffered saline (PBS) is supplemented to a final volume of 4 μL, whole-genome amplification is carried out according to the manufacturer's protocol of single cell genome-wide DNA amplification kit (Vazyme, Discover-sc Single Cell WGA Kit, N603-01).

### 5. PCR Amplification and Detection of the Target Region

52 genome-wide DNA amplification products from the above single blastocyst randomly are selected, 0.5-1 μL for each template is taken, and the following primer pairs are used to preform PCR reaction, respectively, and amplify the expected edited region of myostatin gene in the embryo, and the expected amplification length is about 495 bp.

### Primer Pair for PCR Reaction:

MSTN-F:
SEQ ID NO. 10
5′-TTCCTATGGTTCCTGTAGACTTT-3′,;
MSTN-R:
SEQ ID NO. 11
5′-TTAATTGGAGACATCTTTGTAGG-3′,.

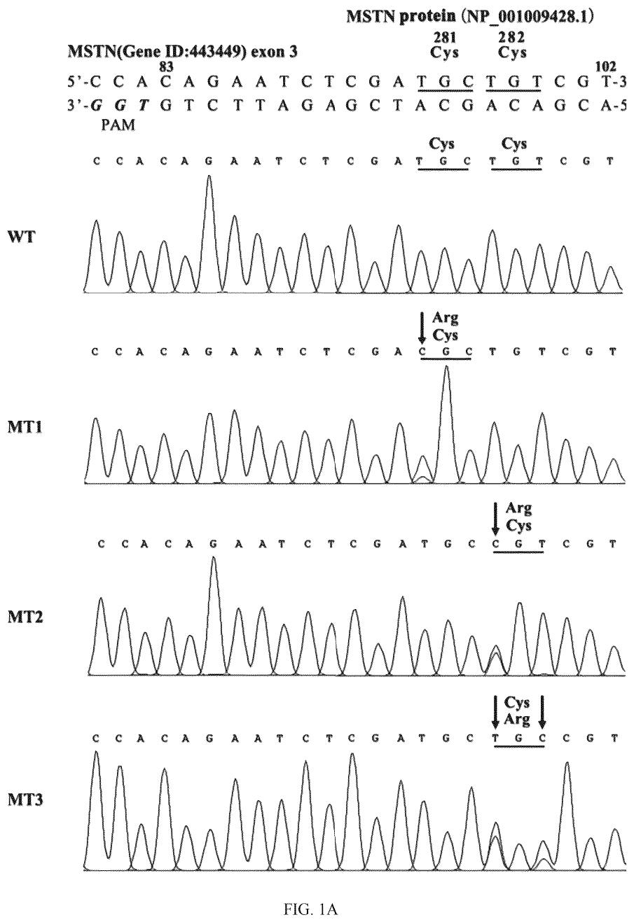

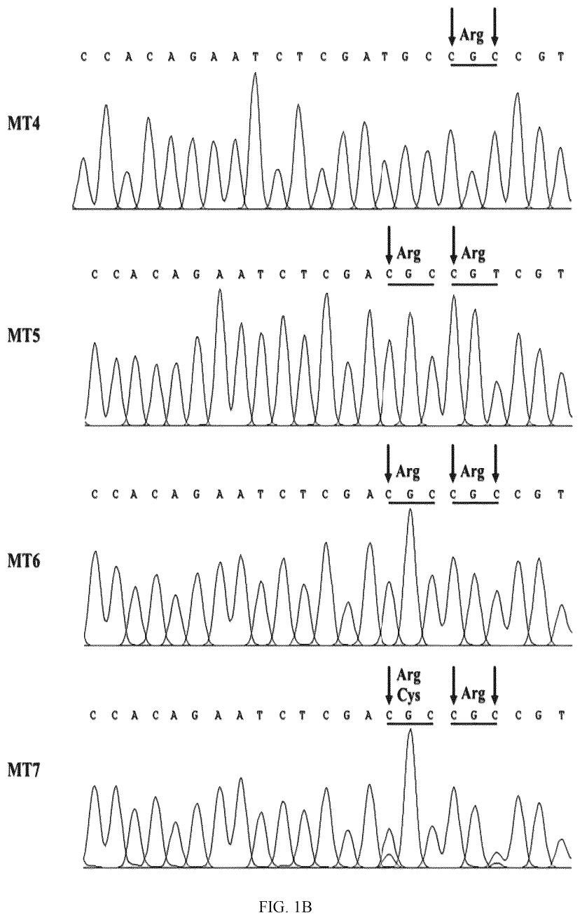

The PCR amplification products of the above 52 embryos are sequenced by MSTN-F and MSTN-R respectively, and compared with the wild-type myostatin gene sequence to identify editing events. Sequencing analysis shows that 39 out of 52 embryos exhibit successful editing at the target site, corresponding to an effective editing efficiency of 75%. Among them: four embryos exhibit a partial T>C mutation at position 841 (chimeric, designated as MT1 in FIG. 1A) within the coding region (SEQ ID NO. 12) of the full-length myostatin gene (Gene ID: 443449) containing the signal peptide, the mutated sequence is shown in SEQ ID NO. 13, this mutation results in the partial substitution of cysteine to arginine at position 281 in the full-length myostatin protein (SEQ ID NO. 14), with the modified sequence presented in SEQ ID NO. 15; 2 embryos show a partial T>C mutation at position 844 (chimeric, designated as MT2 in FIG. 1A) within the coding region of the full-length myostatin gene (Gene ID: 443449) containing the signal peptide, the mutated sequence is shown in SEQ ID NO. 16, resulting in a cysteine-to-arginine partial substitution at position 282 of the full-length myostatin protein, with the mutated sequence presented in SEQ ID NO. 17; 3 embryos exhibit partial T>C mutations at both positions 844 and 846 (chimeric, designated as MT3 in FIG. 1A) in the same gene(Gene ID: 443449) coding region; the mutated sequence is shown in SEQ ID NO. 18, leading to the same Cys282Arg partial substitution in the myostatin protein (SEQ ID NO. 17); 5 embryos have complete T>C mutations at positions 844 and 846 (designated as MT4 in FIG. 1B) in the coding region, the mutated sequence is shown in SEQ ID NO. 18, also resulting in the Cys282Arg substitution in the myostatin protein (SEQ ID NO. 17); 3 embryos display T>C mutations at both positions 841 and 844 (designated as MT5 in FIG. 1B) in the coding region; the mutated sequence is shown in SEQ ID NO. 19, causing cysteine-to-arginine substitutions at both positions 281 and 282 of the myostatin protein (SEQ ID NO. 20); 19 embryos exhibit complete T>C mutations at positions 841, 844, and 846 (designated as MT6 in FIG. 1B) within the coding region (Gene ID: 443449); the mutated sequence is shown in SEQ ID NO. 21, resulting in cysteine-to-arginine substitutions at both positions 281 and 282 of the full-length myostatin protein (SEQ ID NO. 20); 3 embryos show partial T>C mutations at both the positions 841 and 846 and complete T>C mutation at position 844 (designated as MT7 in FIG. 1B) in the coding region; the mutated sequence is shown in SEQ ID NO. 21, also leading to the same Cys281Arg partial substitution and Cys282Arg substitution in the myostatin protein, the modified sequence is shown in SEQ ID NO. 20.

SEQ ID NO. 12:
atgcaaaaactgcaaatctttgtttatatttacctatttatgctgcttgttgctggcccagtggatctgaatgagaacagcgagc
agaaggaaaatgtggaaaaaaaggggctgtgtaatgcatgcttgtggagacaaaacaataaatcctcaagactagaagccataaaaatcca
aatcctcagtaagcttcgcctggaaacagctcctaacatcagcaaagatgctataagacaacttttgcccaaggctcctccactccgggaact
gattgatcagtacgatgtccagagagatgacagcagcgacggctccttggaagacgatgactaccacgttacgacggaaacggtcattacc
atgcccacggagtctgatcttctagcagaagtgcaagaaaaacccaaatgttgcttctttaaatttagctctaagatacaacacaataaagtag
taaaggcccaactgtggatatatctgagacctgtcaagactcctacaacagtgtttgtgcaaatcctgagactcatcaaacccatgaaagacg
gtacaaggtatactggaatccgatctctgaaacttgacatgaacccaggcactggtatttggcagagcattgatgtgaagacagtgttgcaaa
actggctcaaacaacctgaatccaacttaggcattgaaatcaaagctttagatgagaatggtcatgatcttgctgtaaccttcccagaaccagg
agaagaaggactgaatccttttttagaagtcaaggtaacagacacaccaaaaagatctaggagagattttgggcttgattgtgatgagcactc
cacagaatctcgatgctgtcgttaccctctaactgtggattttgaagcttttggatgggattggattattgcacctaaaagatataaggccaat
tactgctctggagaatgtgaatttttatttttgcaaaagtatcctcatacccatcttgtgcaccaagcaaaccccaaaggttcagccggccctt
gctgtactcctacaaagatgtctccaattaatatgctatattttaatggcaaagaacaaataatatatgggaagattccaggcatggtagtaga
tcgctgtgggtgctcatga.
SEQ ID NO. 13:
atgcaaaaactgcaaatctttgtttatatttacctatttatgctgcttgttgctggcccagtggatctgaatgagaacagcgagc
agaaggaaaatgtggaaaaaaaggggctgtgtaatgcatgcttgtggagacaaaacaataaatcctcaagactagaagccataaaaatcca
aatcctcagtaagcttcgcctggaaacagctcctaacatcagcaaagatgctataagacaacttttgcccaaggctcctccactccgggaact
gattgatcagtacgatgtccagagagatgacagcagcgacggctccttggaagacgatgactaccacgttacgacggaaacggtcattacc
atgcccacggagtctgatcttctagcagaagtgcaagaaaaacccaaatgttgcttctttaaatttagctctaagatacaacacaataaagtag
taaaggcccaactgtggatatatctgagacctgtcaagactcctacaacagtgtttgtgcaaatcctgagactcatcaaacccatgaaagacg
gtacaaggtatactggaatccgatctctgaaacttgacatgaacccaggcactggtatttggcagagcattgatgtgaagacagtgttgcaaa
actggctcaaacaacctgaatccaacttaggcattgaaatcaaagctttagatgagaatggtcatgatcttgctgtaaccttcccagaaccagg
agaagaaggactgaatccttttttagaagtcaaggtaacagacacaccaaaaagatctaggagagattttgggcttgattgtgatgagcactc
cacagaatctcgaCgctgtcgttaccctctaactgtggattttgaagcttttggatgggattggattattgcacctaaaagatataaggccaat
tactgctctggagaatgtgaatttttatttttgcaaaagtatcctcatacccatcttgtgcaccaagcaaaccccaaaggttcagccggccctt
gctgtactcctacaaagatgtctccaattaatatgctatattttaatggcaaagaacaaataatatatgggaagattccaggcatggtagtaga
tcgctgtgggtgctcatga.
SEQ ID NO. 14:
MQKLQIFVYIYLFMLLVAGPVDLNENSEQKENVEKKGLCNACLWRQNNKSS
RLEAIKIQILSKLRLETAPNISKDAIRQLLPKAPPLRELIDQYDVQRDDSSDGSLEDDDYH
VTTETVITMPTESDLLAEVQEKPKCCFFKFSSKIQHNKVVKAQLWIYLRPVKTPTTVFVQ
ILRLIKPMKDGTRYTGIRSLKLDMNPGTGIWQSIDVKTVLQNWLKQPESNLGIEIKALDE
NGHDLAVTFPEPGEEGLNPFLEVKVTDTPKRSRRDFGLDCDEHSTESRCCRYPLTVDFE
AFGWDWIIAPKRYKANYCSGECEFLFLQKYPHTHLVHQANPKGSAGPCCTPTKMSPIN
MLYFNGKEQIIYGKIPGMVVDRCGCS.
SEQ ID NO. 15:
MQKLQIFVYIYLFMLLVAGPVDLNENSEQKENVEKKGLCNACLWRQNNKSS
RLEAIKIQILSKLRLETAPNISKDAIRQLLPKAPPLRELIDQYDVQRDDSSDGSLEDDDYH
VTTETVITMPTESDLLAEVQEKPKCCFFKFSSKIQHNKVVKAQLWIYLRPVKTPTTVFVQ
ILRLIKPMKDGTRYTGIRSLKLDMNPGTGIWQSIDVKTVLQNWLKQPESNLGIEIKALDE
NGHDLAVTFPEPGEEGLNPFLEVKVTDTPKRSRRDFGLDCDEHSTESRRCRYPLTVDFE
AFGWDWIIAPKRYKANYCSGECEFLFLQKYPHTHLVHQANPKGSAGPCCTPTKMSPIN
MLYFNGKEQIIYGKIPGMVVDRCGCS.
SEQ ID NO. 16:
atgcaaaaactgcaaatctttgtttatatttacctatttatgctgcttgttgctggcccagtggatctgaatgagaacagcgagc
agaaggaaaatgtggaaaaaaaggggctgtgtaatgcatgcttgtggagacaaaacaataaatcctcaagactagaagccataaaaatcca
aatcctcagtaagcttcgcctggaaacagctcctaacatcagcaaagatgctataagacaacttttgcccaaggctcctccactccgggaact
gattgatcagtacgatgtccagagagatgacagcagcgacggctccttggaagacgatgactaccacgttacgacggaaacggtcattacc
atgcccacggagtctgatcttctagcagaagtgcaagaaaaacccaaatgttgcttctttaaatttagctctaagatacaacacaataaagtag
taaaggcccaactgtggatatatctgagacctgtcaagactcctacaacagtgtttgtgcaaatcctgagactcatcaaacccatgaaagacg
gtacaaggtatactggaatccgatctctgaaacttgacatgaacccaggcactggtatttggcagagcattgatgtgaagacagtgttgcaaa
actggctcaaacaacctgaatccaacttaggcattgaaatcaaagctttagatgagaatggtcatgatcttgctgtaaccttcccagaaccagg
agaagaaggactgaatccttttttagaagtcaaggtaacagacacaccaaaaagatctaggagagattttgggcttgattgtgatgagcactc
cacagaatctcgatgcCgtcgttaccctctaactgtggattttgaagcttttggatgggattggattattgcacctaaaagatataaggccaat
tactgctctggagaatgtgaatttttatttttgcaaaagtatcctcatacccatcttgtgcaccaagcaaaccccaaaggttcagccggccctt
gctgtactcctacaaagatgtctccaattaatatgctatattttaatggcaaagaacaaataatatatgggaagattccaggcatggtagtaga
tcgctgtgggtgctcatga.
SEQ ID NO. 17:
MQKLQIFVYIYLFMLLVAGPVDLNENSEQKENVEKKGLCNACLWRQNNKSS
RLEAIKIQILSKLRLETAPNISKDAIRQLLPKAPPLRELIDQYDVQRDDSSDGSLEDDDYH
VTTETVITMPTESDLLAEVQEKPKCCFFKFSSKIQHNKVVKAQLWIYLRPVKTPTTVFVQ
ILRLIKPMKDGTRYTGIRSLKLDMNPGTGIWQSIDVKTVLQNWLKQPESNLGIEIKALDE
NGHDLAVTFPEPGEEGLNPFLEVKVTDTPKRSRRDFGLDCDEHSTESRCRRYPLTVDFE
AFGWDWIIAPKRYKANYCSGECEFLFLQKYPHTHLVHQANPKGSAGPCCTPTKMSPIN
MLYFNGKEQIIYGKIPGMVVDRCGCS.
SEQ ID NO. 18:
atgcaaaaactgcaaatctttgtttatatttacctatttatgctgcttgttgctggcccagtggatctgaatgagaacagcgagc
agaaggaaaatgtggaaaaaaaggggctgtgtaatgcatgcttgtggagacaaaacaataaatcctcaagactagaagccataaaaatcca
aatcctcagtaagcttcgcctggaaacagctcctaacatcagcaaagatgctataagacaacttttgcccaaggctcctccactccgggaact
gattgatcagtacgatgtccagagagatgacagcagcgacggctccttggaagacgatgactaccacgttacgacggaaacggtcattacc
atgcccacggagtctgatcttctagcagaagtgcaagaaaaacccaaatgttgcttctttaaatttagctctaagatacaacacaataaagtag
taaaggcccaactgtggatatatctgagacctgtcaagactcctacaacagtgtttgtgcaaatcctgagactcatcaaacccatgaaagacg
gtacaaggtatactggaatccgatctctgaaacttgacatgaacccaggcactggtatttggcagagcattgatgtgaagacagtgttgcaaa
actggctcaaacaacctgaatccaacttaggcattgaaatcaaagctttagatgagaatggtcatgatcttgctgtaaccttcccagaaccagg
agaagaaggactgaatccttttttagaagtcaaggtaacagacacaccaaaaagatctaggagagattttgggcttgattgtgatgagcactc
cacagaatctcgatgcCgCcgttaccctctaactgtggattttgaagcttttggatgggattggattattgcacctaaaagatataaggccaat
tactgctctggagaatgtgaatttttatttttgcaaaagtatcctcatacccatcttgtgcaccaagcaaaccccaaaggttcagccggccctt
gctgtactcctacaaagatgtctccaattaatatgctatattttaatggcaaagaacaaataatatatgggaagattccaggcatggtagtaga
tcgctgtgggtgctcatga.
SEQ ID NO. 19:
atgcaaaaactgcaaatctttgtttatatttacctatttatgctgcttgttgctggcccagtggatctgaatgagaacagcgagc
agaaggaaaatgtggaaaaaaaggggctgtgtaatgcatgcttgtggagacaaaacaataaatcctcaagactagaagccataaaaatcca
aatcctcagtaagcttcgcctggaaacagctcctaacatcagcaaagatgctataagacaacttttgcccaaggctcctccactccgggaact
gattgatcagtacgatgtccagagagatgacagcagcgacggctccttggaagacgatgactaccacgttacgacggaaacggtcattacc
atgcccacggagtctgatcttctagcagaagtgcaagaaaaacccaaatgttgcttctttaaatttagctctaagatacaacacaataaagtag
taaaggcccaactgtggatatatctgagacctgtcaagactcctacaacagtgtttgtgcaaatcctgagactcatcaaacccatgaaagacg
gtacaaggtatactggaatccgatctctgaaacttgacatgaacccaggcactggtatttggcagagcattgatgtgaagacagtgttgcaaa
actggctcaaacaacctgaatccaacttaggcattgaaatcaaagctttagatgagaatggtcatgatcttgctgtaaccttcccagaaccagg
agaagaaggactgaatccttttttagaagtcaaggtaacagacacaccaaaaagatctaggagagattttgggcttgattgtgatgagcactc
cacagaatctcgaCgcCgtcgttaccctctaactgtggattttgaagcttttggatgggattggattattgcacctaaaagatataaggccaat
tactgctctggagaatgtgaatttttatttttgcaaaagtatcctcatacccatcttgtgcaccaagcaaaccccaaaggttcagccggccctt
gctgtactcctacaaagatgtctccaattaatatgctatattttaatggcaaagaacaaataatatatgggaagattccaggcatggtagtaga
tcgctgtgggtgctcatga.
SEQ ID NO. 20:
MQKLQIFVYIYLFMLLVAGPVDLNENSEQKENVEKKGLCNACLWRQNNKSS
RLEAIKIQILSKLRLETAPNISKDAIRQLLPKAPPLRELIDQYDVQRDDSSDGSLEDDDYH
VTTETVITMPTESDLLAEVQEKPKCCFFKFSSKIQHNKVVKAQLWIYLRPVKTPTTVFVQ
ILRLIKPMKDGTRYTGIRSLKLDMNPGTGIWQSIDVKTVLQNWLKQPESNLGIEIKALDE
NGHDLAVTFPEPGEEGLNPFLEVKVTDTPKRSRRDFGLDCDEHSTESRRRRYPLTVDFE
AFGWDWIIAPKRYKANYCSGECEFLFLQKYPHTHLVHQANPKGSAGPCCTPTKMSPIN
MLYFNGKEQIIYGKIPGMVVDRCGCS.
SEQ ID NO. 21:
atgcaaaaactgcaaatctttgtttatatttacctatttatgctgcttgttgctggcccagtggatctgaatgagaacagcgagc
agaaggaaaatgtggaaaaaaaggggctgtgtaatgcatgcttgtggagacaaaacaataaatcctcaagactagaagccataaaaatcca
aatcctcagtaagcttcgcctggaaacagctcctaacatcagcaaagatgctataagacaacttttgcccaaggctcctccactccgggaact
gattgatcagtacgatgtccagagagatgacagcagcgacggctccttggaagacgatgactaccacgttacgacggaaacggtcattacc
atgcccacggagtctgatcttctagcagaagtgcaagaaaaacccaaatgttgcttctttaaatttagctctaagatacaacacaataaagtag
taaaggcccaactgtggatatatctgagacctgtcaagactcctacaacagtgtttgtgcaaatcctgagactcatcaaacccatgaaagacg
gtacaaggtatactggaatccgatctctgaaacttgacatgaacccaggcactggtatttggcagagcattgatgtgaagacagtgttgcaaa
actggctcaaacaacctgaatccaacttaggcattgaaatcaaagctttagatgagaatggtcatgatcttgctgtaaccttcccagaaccagg
agaagaaggactgaatccttttttagaagtcaaggtaacagacacaccaaaaagatctaggagagattttgggcttgattgtgatgagcactc
cacagaatctcgaCgcCgCcgttaccctctaactgtggattttgaagcttttggatgggattggattattgcacctaaaagatataaggcca
attactgctctggagaatgtgaatttttatttttgcaaaagtatcctcatacccatcttgtgcaccaagcaaaccccaaaggttcagccggccc
ttgctgtactcctacaaagatgtctccaattaatatgctatattttaatggcaaagaacaaataatatatgggaagattccaggcatggtagta
gatcgctgtgggtgctcatga.

### Embodiment 2 Generation of Hu Sheep with Myostatin Gene Mutations Through ABE Editing

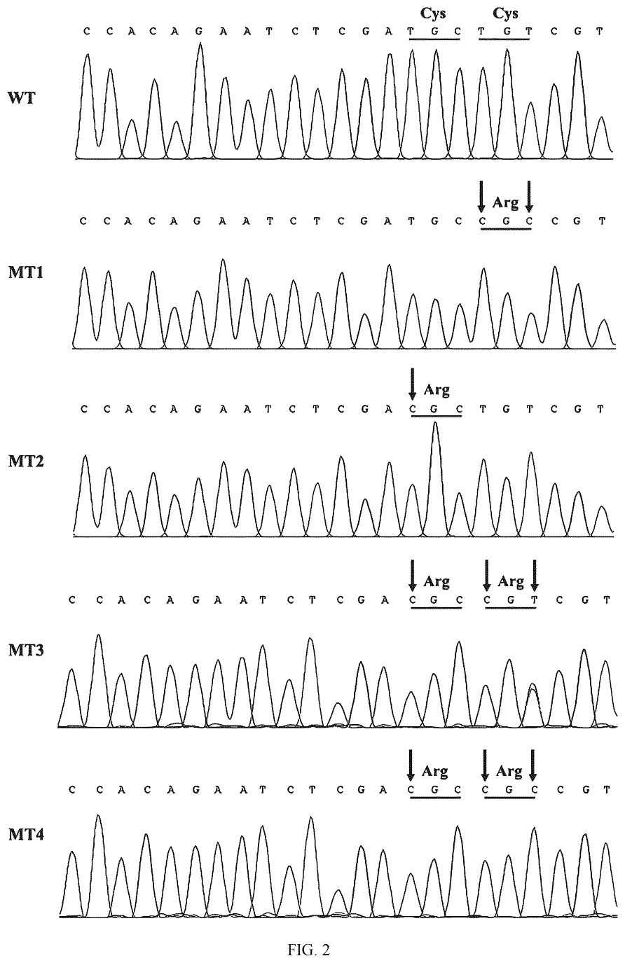

A total of 93 fertilized eggs are obtained from 8 donor ewes (Hu sheep), and the mixed system (sgRNA+ABE mRNA) obtained in the third part of Embodiment 1 is injected into the fertilized eggs cytoplasm via microinjection. The injection amount of each fertilized egg is 5 pL, the final concentrations in the mixture are 50 ng/μL for sgRNA and 100 ng/μL for ABE mRNA; A total of 72 morphologically normal fertilized eggs following microinjection are transplanted into 14 surrogate ewes, yielding 11 live lambs at parturition. Genomic DNA is extracted from lamb ear tissue samples, and PCR amplification is carried out by using MSTN-F+MSTN-R primer pair in Embodiment 1, and the PCR products are sent to sequencing companies for bidirectional sequencing with MSTN-F and MSTN-R primers respectively. The results show that two lambs exhibit T>C mutation at the 841st in the coding region of myostatin full length gene (Gene ID: 443449) containing signal peptide, cysteine at position 281 of myostatin full length protein is mutated into arginine (MT2 in FIG. 2); two lambs exhibit T>C mutations at both the 844th and 846th positions in the coding region of myostatin full length gene (Gene ID: 443449) containing signal peptide, resulting in cysteine-to-arginine substitutions at position 282 of myostatin full length protein (MT1 in FIG. 2); one lamb exhibits T>C mutations at both the 841st and 844th positions and partial T>C mutations at the 846th position (chimeric) in the coding region of myostatin full length gene (Gene ID: 443449) containing signal peptide, causing cysteine-to-arginine substitutions at both positions 281 and 282 of myostatin full length protein (MT3 in FIG. 2); three lambs exhibit complete T>C mutations at the 841st, 844th and 846th position in the coding regions of myostatin full length gene (Gene ID: 443449) containing signal peptide, also leading to the same Cys281Arg and Cys282Arg substitutions in the myostatin protein (MT4 in FIG. 2), the positive rate of gene editing is 72.7%.

### Embodiment 3 Phenotypic Analysis of Myostatin Gene Mutant Hu Sheep

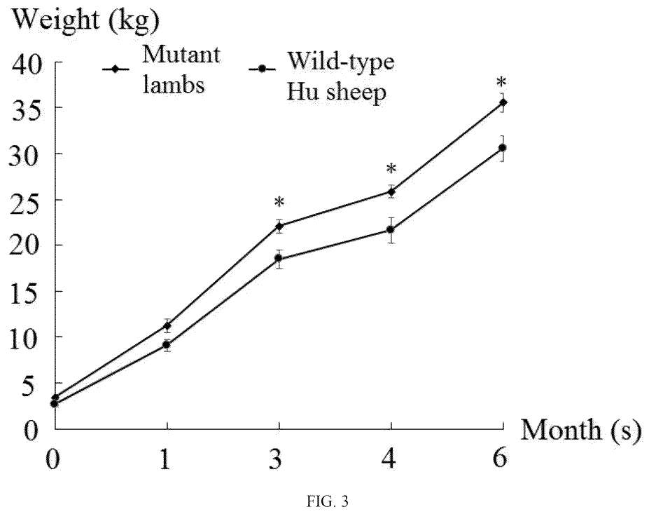

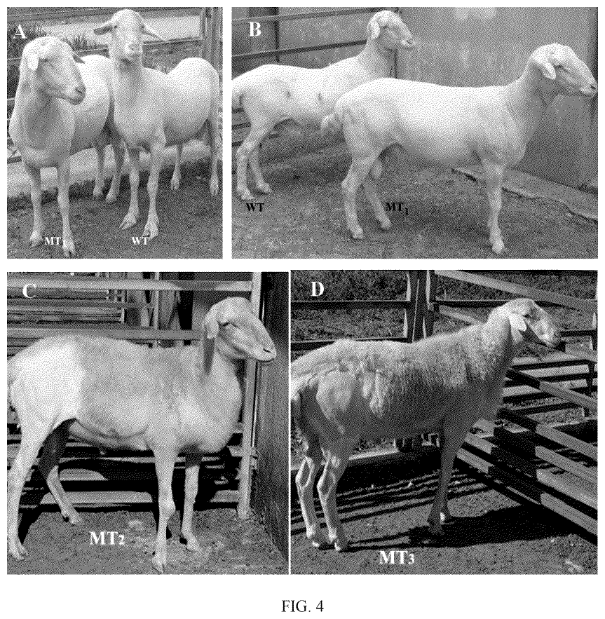

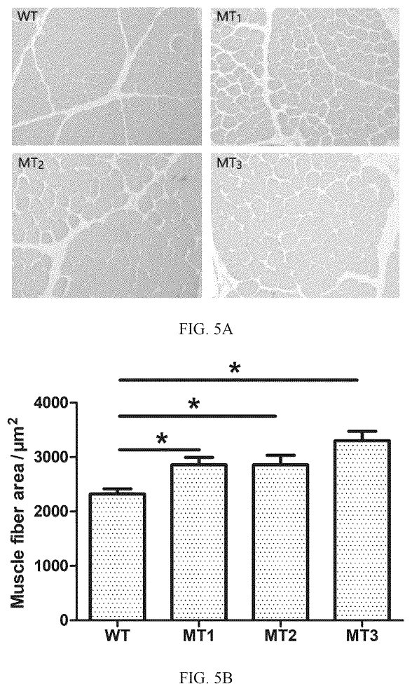

Both the myostatin-mutant lambs obtained from Embodiment 2 and wild-type Hu sheep are raised under the same conditions and subjected to phenotypic observation. The results show that the body weights of myostatin protein mutant lambs are significantly higher than those of wild-type Hu sheep at 3, 4, and 6 months of age (FIG. 3). Comparative analysis of 7-month-old myostatin-mutant Hu sheep and age-matched wild-type Hu sheep under identical husbandry conditions shows that the myostatin mutants exhibit significantly more developed and voluminous gluteal and forelimb muscles (as shown in FIG. 4), displaying a distinct double-muscling phenotype. Surgical biopsy samples of gluteal muscles are collected from both myostatin-mutant and wild-type sheep, followed by fixation, histological sectioning, and HE staining (as shown in FIG. 5A). The results show that myostatin-mutant sheep (MT1-MT3 in FIG. 5B) have markedly increased muscle fiber cross-sectional area in the gluteal muscles compared to wild-type controls (WT in FIG. 5B).

### Embodiment 4 Preparation of Sheep Fibroblasts Carrying Myostatin Protein Mutant by ABE Editing

Sheep fibroblasts are co-transfected with the recombinant vector pGL3-sg and the ABE8e vector. Forty-eight hours post-transfection, puromycin selection is applied for 7 days to establish stable cell lines. Single-cell cloning and expansion are subsequently performed, yielding a total of 18 monoclonal cell lines. Genomic DNA is extracted from monoclonal cells, and PCR amplification is carried out by using MSTN-F+MSTN-R primer pair. The amplified products are sequenced by MSTN-F and MSTN-R respectively, and compared with the wild-type myostatin gene sequence. The analysis shows that 13 monoclonal cells are effectively edited, where two clones show a T>C mutation at position 841 in the coding region of the full-length myostatin gene containing the signal peptide (Gene ID: 443449, SEQ ID NO. 12), the mutated sequence is shown in SEQ ID NO. 13, and the cysteine at the 281st position of myostatin full length protein is mutated into arginine as shown in SEQ ID NO. 15; two clones exhibit T>C mutations at both positions 844 and 846 within the coding region of the myostatin full length gene (Gene ID: 443449) containing signal peptide, the mutated sequence is shown in SEQ ID NO. 18, and the cysteine at the 282nd position of myostatin full length protein is mutated into arginine as shown in SEQ ID NO. 17; nine clones have T>C mutations at positions 841, 844, and 846 in the coding region, the mutated sequence is shown in SEQ ID NO. 21, causing cysteine-to-arginine substitutions at both positions 281 and 282 of the myostatin protein (SEQ ID NO. 20).

The above-mentioned embodiments only describe the preferred mode of the disclosure, and do not limit the scope of the disclosure. Under the premise of not departing from the design spirit of the disclosure, various modifications and improvements made by ordinary technicians in the field to the technical scheme of the disclosure shall fall within the protection scope determined by the claims of the disclosure.

What is claimed is:

1. A myostatin (MSTN) protein mutant, wherein an amino acid sequence of the myostatin protein mutant is any one of sequences shown in SEQ ID NO. 15, SEQ ID NO. 17 or SEQ ID NO. 20.

2. The myostatin protein mutant according to claim 1, wherein when the amino acid sequence of the myostatin protein mutant is SEQ ID NO. 15, a nucleotide sequence of a coding gene is shown in SEQ ID NO. 13; when the amino acid sequence of the myostatin protein mutant is SEQ ID NO. 17, the nucleotide sequence of the coding gene is shown in SEQ ID NO. 16 or SEQ ID NO. 18; and when the amino acid sequence of the myostatin protein mutant is SEQ ID NO. 20, the nucleotide sequence of the coding gene is shown in SEQ ID NO. 19 or SEQ ID NO. 21.

3. A method for preparing a biological material, wherein a genome comprises a gene encoding the myostatin protein mutant according to claim 1, comprising a step of obtaining the biological material through adenine base editor (ABE) editing under a guidance of a single guide ribonucleic acid (sgRNA); and
wherein a nucleotide sequence of the sgRNA is shown in SEQ ID NO. 5.

4. The method according to claim 3, wherein the biological material comprises cells, embryos and animals.

5. A method for increasing animal muscle yield or promoting animal muscle development, comprising using a single guide ribonucleic acid (sgRNA) to mutate a myostatin coding gene in an animal genome into the coding gene according to claim 2 through adenine base editor (ABE) editing technology; and
wherein a nucleotide sequence of the sgRNA is shown in SEQ ID NO. 5.


SEQUENCE LISTING (21 sequences)

SEQ ID NO: 1 - DNA, 24 bp, synthetic construct
  source 1..24: mol_type = other DNA
ccggacgacagcatcgagattctg

SEQ ID NO: 2 - DNA, 24 bp, synthetic construct
  source 1..24: mol_type = other DNA
aaaccagaatctcgatgctgtcgt

SEQ ID NO: 3 - DNA, 39 bp, synthetic construct
  source 1..39: mol_type = other DNA
taatacgactcactataggacgacagcatcgagattctg

SEQ ID NO: 4 - DNA, 21 bp, synthetic construct
  source 1..21: mol_type = other DNA
aaaagcaccgactcggtgcca

SEQ ID NO: 5 - RNA, 96 nt, synthetic construct
  source 1..96: mol_type = other RNA
acgacagcatcgagattctggttttagagctagaaatagcaagttaaaataaggctagtccgttatcaacttgaaaaagtggcaccgagtcggtgc

SEQ ID NO: 6 - DNA, 20 bp, synthetic construct
  source 1..20: mol_type = other DNA
acgacagcatcgagattctg

SEQ ID NO: 7 - DNA, 25 bp, synthetic construct
  source 1..25: mol_type = other DNA
ctaatacgactcactatagggagag

SEQ ID NO: 8 - DNA, 18 bp, synthetic construct
  source 1..18: mol_type = other DNA
aaggcacagtcgaggctg

SEQ ID NO: 9 - RNA, 4818 nt, synthetic construct
  source 1..4818: mol_type = other RNA
atgaaacggacagccgacggaagcgagttcgagtcaccaaagaagaagcggaaagtctctgaggtggagttttcccacgagtactggatgagacatgccctgaccctggccaagagggcacgggatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgattgacgccaccctgtacgtgacattcgagccttgcgtgatgtgcgccggcgccatgatccactctaggatcggccgcgtggtgtttggcgtgaggaactcaaaaagaggcgccgcaggctccctgatgaacgtgctgaactaccccggcatgaatcaccgcgtcgaaattaccgagggaatcctggcagatgaatgtgccgccctgctgtgcgatttctatcggatgcctagacaggtgttcaatgctcagaagaaggcccagagctccatcaactccggaggatctagcggaggctcctctggctctgagacacctggcacaagcgagagcgcaacacctgaaagcagcgggggcagcagcggggggtcagacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgactctggcggctcaaaaagaaccgccgacggcagcgaattcgagcccaagaagaagaggaaagtctaa

SEQ ID NO: 10 - DNA, 23 bp, synthetic construct
  source 1..23: mol_type = other DNA
ttcctatggttcctgtagacttt

SEQ ID NO: 11 - DNA, 23 bp, synthetic construct
  source 1..23: mol_type = other DNA
ttaattggagacatctttgtagg

SEQ ID NO: 12 - DNA, 1128 bp, Ovis aries
  source 1..1128: mol_type = other DNA
atgcaaaaactgcaaatctttgtttatatttacctatttatgctgcttgttgctggcccagtggatctgaatgagaacagcgagcagaaggaaaatgtggaaaaaaaggggctgtgtaatgcatgcttgtggagacaaaacaataaatcctcaagactagaagccataaaaatccaaatcctcagtaagcttcgcctggaaacagctcctaacatcagcaaagatgctataagacaacttttgcccaaggctcctccactccgggaactgattgatcagtacgatgtccagagagatgacagcagcgacggctccttggaagacgatgactaccacgttacgacggaaacggtcattaccatgcccacggagtctgatcttctagcagaagtgcaagaaaaacccaaatgttgcttctttaaatttagctctaagatacaacacaataaagtagtaaaggcccaactgtggatatatctgagacctgtcaagactcctacaacagtgtttgtgcaaatcctgagactcatcaaacccatgaaagacggtacaaggtatactggaatccgatctctgaaacttgacatgaacccaggcactggtatttggcagagcattgatgtgaagacagtgttgcaaaactggctcaaacaacctgaatccaacttaggcattgaaatcaaagctttagatgagaatggtcatgatcttgctgtaaccttcccagaaccaggagaagaaggactgaatccttttttagaagtcaaggtaacagacacaccaaaaagatctaggagagattttgggcttgattgtgatgagcactccacagaatctcgatgctgtcgttaccctctaactgtggattttgaagcttttggatgggattggattattgcacctaaaagatataaggccaattactgctctggagaatgtgaatttttatttttgcaaaagtatcctcatacccatcttgtgcaccaagcaaaccccaaaggttcagccggcccttgctgtactcctacaaagatgtctccaattaatatgctatattttaatggcaaagaacaaataatatatgggaagattccaggcatggtagtagatcgctgtgggtgctcatga

SEQ ID NO: 13 - DNA, 1128 bp, synthetic construct
  source 1..1128: mol_type = other DNA
atgcaaaaactgcaaatctttgtttatatttacctatttatgctgcttgttgctggcccagtggatctgaatgagaacagcgagcagaaggaaaatgtggaaaaaaaggggctgtgtaatgcatgcttgtggagacaaaacaataaatcctcaagactagaagccataaaaatccaaatcctcagtaagcttcgcctggaaacagctcctaacatcagcaaagatgctataagacaacttttgcccaaggctcctccactccgggaactgattgatcagtacgatgtccagagagatgacagcagcgacggctccttggaagacgatgactaccacgttacgacggaaacggtcattaccatgcccacggagtctgatcttctagcagaagtgcaagaaaaacccaaatgttgcttctttaaatttagctctaagatacaacacaataaagtagtaaaggcccaactgtggatatatctgagacctgtcaagactcctacaacagtgtttgtgcaaatcctgagactcatcaaacccatgaaagacggtacaaggtatactggaatccgatctctgaaacttgacatgaacccaggcactggtatttggcagagcattgatgtgaagacagtgttgcaaaactggctcaaacaacctgaatccaacttaggcattgaaatcaaagctttagatgagaatggtcatgatcttgctgtaaccttcccagaaccaggagaagaaggactgaatccttttttagaagtcaaggtaacagacacaccaaaaagatctaggagagattttgggcttgattgtgatgagcactccacagaatctcgacgctgtcgttaccctctaactgtggattttgaagcttttggatgggattggattattgcacctaaaagatataaggccaattactgctctggagaatgtgaatttttatttttgcaaaagtatcctcatacccatcttgtgcaccaagcaaaccccaaaggttcagccggcccttgctgtactcctacaaagatgtctccaattaatatgctatattttaatggcaaagaacaaataatatatgggaagattccaggcatggtagtagatcgctgtgggtgctcatga

SEQ ID NO: 14 - protein, 375 aa, Ovis aries
MQKLQIFVYIYLFMLLVAGPVDLNENSEQKENVEKKGLCNACLWRQNNKSSRLEAIKIQILSKLRLETAPNISKDAIRQLLPKAPPLRELIDQYDVQRDDSSDGSLEDDDYHVTTETVITMPTESDLLAEVQEKPKCCFFKFSSKIQHNKVVKAQLWIYLRPVKTPTTVFVQILRLIKPMKDGTRYTGIRSLKLDMNPGTGIWQSIDVKTVLQNWLKQPESNLGIEIKALDENGHDLAVTFPEPGEEGLNPFLEVKVTDTPKRSRRDFGLDCDEHSTESRCCRYPLTVDFEAFGWDWIIAPKRYKANYCSGECEFLFLQKYPHTHLVHQANPKGSAGPCCTPTKMSPINMLYFNGKEQIIYGKIPGMVVDRCGCS

SEQ ID NO: 15 - protein, 375 aa, synthetic construct
MQKLQIFVYIYLFMLLVAGPVDLNENSEQKENVEKKGLCNACLWRQNNKSSRLEAIKIQILSKLRLETAPNISKDAIRQLLPKAPPLRELIDQYDVQRDDSSDGSLEDDDYHVTTETVITMPTESDLLAEVQEKPKCCFFKFSSKIQHNKVVKAQLWIYLRPVKTPTTVFVQILRLIKPMKDGTRYTGIRSLKLDMNPGTGIWQSIDVKTVLQNWLKQPESNLGIEIKALDENGHDLAVTFPEPGEEGLNPFLEVKVTDTPKRSRRDFGLDCDEHSTESRRCRYPLTVDFEAFGWDWIIAPKRYKANYCSGECEFLFLQKYPHTHLVHQANPKGSAGPCCTPTKMSPINMLYFNGKEQIIYGKIPGMVVDRCGCS

SEQ ID NO: 16 - DNA, 1128 bp, synthetic construct
  source 1..1128: mol_type = other DNA
atgcaaaaactgcaaatctttgtttatatttacctatttatgctgcttgttgctggcccagtggatctgaatgagaacagcgagcagaaggaaaatgtggaaaaaaaggggctgtgtaatgcatgcttgtggagacaaaacaataaatcctcaagactagaagccataaaaatccaaatcctcagtaagcttcgcctggaaacagctcctaacatcagcaaagatgctataagacaacttttgcccaaggctcctccactccgggaactgattgatcagtacgatgtccagagagatgacagcagcgacggctccttggaagacgatgactaccacgttacgacggaaacggtcattaccatgcccacggagtctgatcttctagcagaagtgcaagaaaaacccaaatgttgcttctttaaatttagctctaagatacaacacaataaagtagtaaaggcccaactgtggatatatctgagacctgtcaagactcctacaacagtgtttgtgcaaatcctgagactcatcaaacccatgaaagacggtacaaggtatactggaatccgatctctgaaacttgacatgaacccaggcactggtatttggcagagcattgatgtgaagacagtgttgcaaaactggctcaaacaacctgaatccaacttaggcattgaaatcaaagctttagatgagaatggtcatgatcttgctgtaaccttcccagaaccaggagaagaaggactgaatccttttttagaagtcaaggtaacagacacaccaaaaagatctaggagagattttgggcttgattgtgatgagcactccacagaatctcgatgccgtcgttaccctctaactgtggattttgaagcttttggatgggattggattattgcacctaaaagatataaggccaattactgctctggagaatgtgaatttttatttttgcaaaagtatcctcatacccatcttgtgcaccaagcaaaccccaaaggttcagccggcccttgctgtactcctacaaagatgtctccaattaatatgctatattttaatggcaaagaacaaataatatatgggaagattccaggcatggtagtagatcgctgtgggtgctcatga

SEQ ID NO: 17 - protein, 375 aa, synthetic construct
MQKLQIFVYIYLFMLLVAGPVDLNENSEQKENVEKKGLCNACLWRQNNKSSRLEAIKIQILSKLRLETAPNISKDAIRQLLPKAPPLRELIDQYDVQRDDSSDGSLEDDDYHVTTETVITMPTESDLLAEVQEKPKCCFFKFSSKIQHNKVVKAQLWIYLRPVKTPTTVFVQILRLIKPMKDGTRYTGIRSLKLDMNPGTGIWQSIDVKTVLQNWLKQPESNLGIEIKALDENGHDLAVTFPEPGEEGLNPFLEVKVTDTPKRSRRDFGLDCDEHSTESRCRRYPLTVDFEAFGWDWIIAPKRYKANYCSGECEFLFLQKYPHTHLVHQANPKGSAGPCCTPTKMSPINMLYFNGKEQIIYGKIPGMVVDRCGCS

SEQ ID NO: 18 - DNA, 1128 bp, synthetic construct
  source 1..1128: mol_type = other DNA
atgcaaaaactgcaaatctttgtttatatttacctatttatgctgcttgttgctggcccagtggatctgaatgagaacagcgagcagaaggaaaatgtggaaaaaaaggggctgtgtaatgcatgcttgtggagacaaaacaataaatcctcaagactagaagccataaaaatccaaatcctcagtaagcttcgcctggaaacagctcctaacatcagcaaagatgctataagacaacttttgcccaaggctcctccactccgggaactgattgatcagtacgatgtccagagagatgacagcagcgacggctccttggaagacgatgactaccacgttacgacggaaacggtcattaccatgcccacggagtctgatcttctagcagaagtgcaagaaaaacccaaatgttgcttctttaaatttagctctaagatacaacacaataaagtagtaaaggcccaactgtggatatatctgagacctgtcaagactcctacaacagtgtttgtgcaaatcctgagactcatcaaacccatgaaagacggtacaaggtatactggaatccgatctctgaaacttgacatgaacccaggcactggtatttggcagagcattgatgtgaagacagtgttgcaaaactggctcaaacaacctgaatccaacttaggcattgaaatcaaagctttagatgagaatggtcatgatcttgctgtaaccttcccagaaccaggagaagaaggactgaatccttttttagaagtcaaggtaacagacacaccaaaaagatctaggagagattttgggcttgattgtgatgagcactccacagaatctcgatgccgccgttaccctctaactgtggattttgaagcttttggatgggattggattattgcacctaaaagatataaggccaattactgctctggagaatgtgaatttttatttttgcaaaagtatcctcatacccatcttgtgcaccaagcaaaccccaaaggttcagccggcccttgctgtactcctacaaagatgtctccaattaatatgctatattttaatggcaaagaacaaataatatatgggaagattccaggcatggtagtagatcgctgtgggtgctcatga

SEQ ID NO: 19 - DNA, 1128 bp, synthetic construct
  source 1..1128: mol_type = other DNA
atgcaaaaactgcaaatctttgtttatatttacctatttatgctgcttgttgctggcccagtggatctgaatgagaacagcgagcagaaggaaaatgtggaaaaaaaggggctgtgtaatgcatgcttgtggagacaaaacaataaatcctcaagactagaagccataaaaatccaaatcctcagtaagcttcgcctggaaacagctcctaacatcagcaaagatgctataagacaacttttgcccaaggctcctccactccgggaactgattgatcagtacgatgtccagagagatgacagcagcgacggctccttggaagacgatgactaccacgttacgacggaaacggtcattaccatgcccacggagtctgatcttctagcagaagtgcaagaaaaacccaaatgttgcttctttaaatttagctctaagatacaacacaataaagtagtaaaggcccaactgtggatatatctgagacctgtcaagactcctacaacagtgtttgtgcaaatcctgagactcatcaaacccatgaaagacggtacaaggtatactggaatccgatctctgaaacttgacatgaacccaggcactggtatttggcagagcattgatgtgaagacagtgttgcaaaactggctcaaacaacctgaatccaacttaggcattgaaatcaaagctttagatgagaatggtcatgatcttgctgtaaccttcccagaaccaggagaagaaggactgaatccttttttagaagtcaaggtaacagacacaccaaaaagatctaggagagattttgggcttgattgtgatgagcactccacagaatctcgacgccgtcgttaccctctaactgtggattttgaagcttttggatgggattggattattgcacctaaaagatataaggccaattactgctctggagaatgtgaatttttatttttgcaaaagtatcctcatacccatcttgtgcaccaagcaaaccccaaaggttcagccggcccttgctgtactcctacaaagatgtctccaattaatatgctatattttaatggcaaagaacaaataatatatgggaagattccaggcatggtagtagatcgctgtgggtgctcatga

SEQ ID NO: 20 - protein, 375 aa, synthetic construct
MQKLQIFVYIYLFMLLVAGPVDLNENSEQKENVEKKGLCNACLWRQNNKSSRLEAIKIQILSKLRLETAPNISKDAIRQLLPKAPPLRELIDQYDVQRDDSSDGSLEDDDYHVTTETVITMPTESDLLAEVQEKPKCCFFKFSSKIQHNKVVKAQLWIYLRPVKTPTTVFVQILRLIKPMKDGTRYTGIRSLKLDMNPGTGIWQSIDVKTVLQNWLKQPESNLGIEIKALDENGHDLAVTFPEPGEEGLNPFLEVKVTDTPKRSRRDFGLDCDEHSTESRRRRYPLTVDFEAFGWDWIIAPKRYKANYCSGECEFLFLQKYPHTHLVHQANPKGSAGPCCTPTKMSPINMLYFNGKEQIIYGKIPGMVVDRCGCS

SEQ ID NO: 21 - DNA, 1128 bp, synthetic construct
  source 1..1128: mol_type = other DNA
atgcaaaaactgcaaatctttgtttatatttacctatttatgctgcttgttgctggcccagtggatctgaatgagaacagcgagcagaaggaaaatgtggaaaaaaaggggctgtgtaatgcatgcttgtggagacaaaacaataaatcctcaagactagaagccataaaaatccaaatcctcagtaagcttcgcctggaaacagctcctaacatcagcaaagatgctataagacaacttttgcccaaggctcctccactccgggaactgattgatcagtacgatgtccagagagatgacagcagcgacggctccttggaagacgatgactaccacgttacgacggaaacggtcattaccatgcccacggagtctgatcttctagcagaagtgcaagaaaaacccaaatgttgcttctttaaatttagctctaagatacaacacaataaagtagtaaaggcccaactgtggatatatctgagacctgtcaagactcctacaacagtgtttgtgcaaatcctgagactcatcaaacccatgaaagacggtacaaggtatactggaatccgatctctgaaacttgacatgaacccaggcactggtatttggcagagcattgatgtgaagacagtgttgcaaaactggctcaaacaacctgaatccaacttaggcattgaaatcaaagctttagatgagaatggtcatgatcttgctgtaaccttcccagaaccaggagaagaaggactgaatccttttttagaagtcaaggtaacagacacaccaaaaagatctaggagagattttgggcttgattgtgatgagcactccacagaatctcgacgccgccgttaccctctaactgtggattttgaagcttttggatgggattggattattgcacctaaaagatataaggccaattactgctctggagaatgtgaatttttatttttgcaaaagtatcctcatacccatcttgtgcaccaagcaaaccccaaaggttcagccggcccttgctgtactcctacaaagatgtctccaattaatatgctatattttaatggcaaagaacaaataatatatgggaagattccaggcatggtagtagatcgctgtgggtgctcatga